(19) United States
(12) Patent Application Publication
(10) Pub. No.: US 2020/0305695 A1
(43) Pub. Date: Oct. 1, 2020

(54) RIGID ENDOSCOPE SYSTEM

(71) Applicant: JVCKENWOOD CORPORATION, Yokohama-shi (JP)

(72) Inventor: Hideki TENGEIJI, Yokohama-shi (JP)

(73) Assignee: JVCKENWOOD Corporation

(21) Appl. No.: 16/810,654

(22) Filed: Mar. 5, 2020

(30) Foreign Application Priority Data
Mar. 25, 2019 (JP) 2019-056064

(51) Int. Cl. A61B 1/00; H04N 5/235; A61B 1/04; A61B 1/06; A61B 5/00

(52) U.S. Cl. CPC A61B 1/00188; H04N 5/2352; A61B 1/00078; A61B 1/042; H04N 2005/2255; A61B 1/0669; A61B 1/00009; A61B 1/00006; A61B 5/0086; A61B 1/00096

(57) ABSTRACT

A rigid endoscope system includes: a pipe body; an objective lens; an ocular lens; an image pick-up sensor; a light source that emits a detection light to an object to be observed; a light receiving sensor that receives the detection light reflected by the object to be observed and has passed through the inside of the pipe body; a spectroscopic device that spectrally disperses a luminous flux emitted from the ocular lens in each of a direction toward the image pick-up sensor and a direction toward the light receiving sensor; a unit that calculates a distance to the object to be observed; an image-forming system that forms an image of a portion of the luminous flux on the image pick-up sensor; and a focus adjustment unit that performs a focus adjustment by moving a lens included in the image-forming system in an optical axis direction based on the calculated distance.

### CROSS REFERENCE TO RELATED APPLICATION

This application is based upon and claims the benefit of priority from Japanese patent application No. 2019-056064, filed on Mar. 25, 2019, the disclosure of which is incorporated herein in its entirety by reference.

### BACKGROUND

The present disclosure relates to a rigid endoscope system.

A rigid endoscope including an insertion part that does not have flexibility is known. The rigid endoscope transmits an observation image formed by an objective optical system disposed at a tip end of the insertion part to a substrate edge side by a relay optical system. The observation image transmitted to the substrate edge side is formed on a light receiving surface of an image pick-up device and converted to an image signal to be output (e.g., Japanese Unexamined Patent Application Publication No. H8-122667).

### SUMMARY

A rigid endoscope system which transmits a high quality output image is being demanded due to the progress in medical care, and thus there is a tendency to employ a large scale image pick-up device of high pixel count for the rigid endoscope system. When the scale of the image pick-up device is enlarged, the depth of the image becomes shallow, thus requiring a strict focusing adjustment, and hence it is difficult to perform fast focusing adjustment. On the other hand, since an outer diameter of a pipe body that is inserted into a body of a test subject is small, an F value of the entire optical system is large, which, for instance, is disadvantageous for a contrast AF that utilizes an observation image output from the image pick-up device.

A rigid endoscope system according to an aspect of the present disclosure includes: a linear pipe body; an objective lens provided at one end side of the pipe body; an ocular lens provided at the other end side of the pipe body opposite to the one end side thereof; an image pick-up sensor configured to output an image pick-up signal for generating image data; a detection light source configured to emit a detection light to an object to be observed; a light receiving sensor configured to receive the detection light that is reflected by the object to be observed and has passed through the inside of the pipe body; a spectroscopic optical device configured to spectrally disperse a luminous flux emitted from the ocular lens in each of a direction toward the image pick-up sensor and a direction toward the light receiving sensor; a calculation unit configured to calculate a distance to the object to be observed by measuring time taken from emission of the detection light from the detection light source to reception thereof by the light receiving sensor; an image-forming optical system configured to form an image of a portion of the luminous flux emitted from the ocular lens on the image pick-up sensor; and a focus adjustment unit configured to perform a focus adjustment by moving a focus adjustment lens included in the image-forming optical system in an optical axis direction based on the distance calculated by the calculation unit.

### BRIEF DESCRIPTION OF THE DRAWINGS

The above and other aspects, advantages and features will be more apparent from the following description of certain embodiments taken in conjunction with the accompanying drawings, in which:

FIG. 1 is an overall schematic diagram of a rigid endoscope system according to a first embodiment;

FIG. 2 is a diagram showing a state in which an optical system is accommodated inside a pipe body;

FIG. 3 is an overall schematic diagram of a rigid endoscope system according to a second embodiment;

FIG. 4 is an overall schematic diagram of a rigid endoscope system according to a third embodiment; and

FIG. 5 is an overall schematic diagram of a rigid endoscope system according to a fourth embodiment.

### DETAILED DESCRIPTION

Hereinbelow, the present disclosure is explained through embodiments, however, the disclosure of the claims is not to be limited to the embodiments mentioned below. Further, not all of the structures explained in the embodiments are necessary as a means for solving the problem.

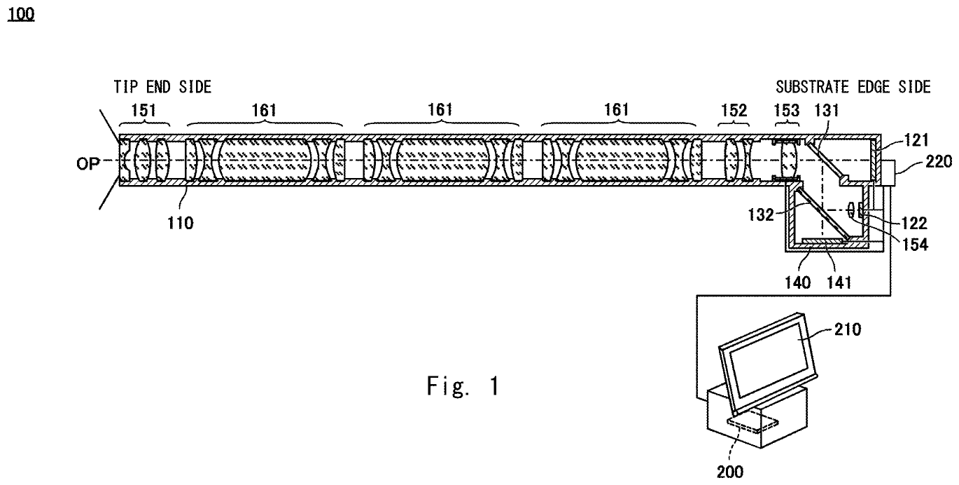

FIG. 1 is an overall schematic diagram of a rigid endoscope system 100 according to a first embodiment. The rigid endoscope system 100 includes, in appearance, a pipe body 110 extending linearly, a control unit 200 connected thereto via a cable, and a display unit 210 as the main structural components. In the figure, the pipe body 110 is shown in a cross section and the main components that are accommodated in the pipe body 110 are shown.

The pipe body 110 is, for example, a stainless pipe and does have flexibility. The pipe body 110 extends linearly from one end which is a tip end side pointed toward an object to be observed to the other end which is a substrate edge side, and includes a branched pipe 140 that braches from the pipe body 110 on the substrate edge side.

In this embodiment, a central axis of the pipe body 110 coincides with an optical axis OP of a main optical system. A plurality of lenses are disposed inside the pipe body 110. Specifically, an objective lens 151, a relay lens 161, an ocular lens 152, and an image-forming lens 153 are disposed in this order from the tip end side toward the substrate edge side. An image pick-up sensor 121 is disposed to the other end of the pipe body 110.

The objective lens 151 forms an observation image of the object to be observed, and the relay lens 161 transmits the observation image formed by the objective lens 151 to the ocular lens 152. Each of the objective lens 151, the relay lens 161, and the ocular lens 152 may be configured of a plurality of lenses. For example, the objective lens 151 is configured of three lenses of negative, positive, and positive lenses. Further, a pair of relay lenses 161 is configured of five lenses of positive, negative, positive, negative, and positive lenses symmetrically arranged in an optical axis direction. Specifically, a rod lens having a length longer than a diameter in the optical axis direction may be employed for the third lens which is a positive lens in order to ensure a relay length. Further, plural pairs of relay lenses 161 may be disposed in accordance with the length of the pipe body 110. In the present embodiment shown in FIG. 1, three pairs of relay lenses 161 are disposed.

The image-forming lens 153 has a function as an image-forming optical system of re-imaging the observation image that has passed through the ocular lens 152 on a light receiving surface of the image pick-up sensor 121 in accordance with the size of the light receiving surface. In the figure, the image-forming lens 153 is shown as one lens, however, it may be configured of a plurality of lenses. When the image-forming lens 153 is configured of a plurality of lenses as an image-forming optical system, at least a part of the lenses is configured as a focus adjustment lens that is moveable in the optical axis OP direction by being driven by an actuator. When the image-forming optical system is configured solely of the image-forming lens 153, the image-forming lens 153 is configured as the focus adjustment lens that is moveable in the optical axis OP direction by being driven by the actuator.

The image pick-up sensor 121 is, for example, a CMOS sensor, and photoelectronically converts the optical image formed on the light receiving surface and outputs the converted image as an image pick-up signal. A circuit board 220 disposed to a substrate edge part of the pipe body 110 adjusts a level of the image pick-up signal output from the image pick-up sensor 121 for A/D conversion and transmits the converted signal as pixel data to the control unit 200.

A half mirror 131 is disposed between the image-forming lens 153 and the image pick-up sensor 121 in an oblique manner with respect to the optical axis OP. The half mirror 131 is a spectroscopic optical device that transmits a portion of luminous flux emitted from the image-forming lens 153 to the image pick-up sensor 121 side and reflects the rest of the luminous flux. A light source 141 is disposed at a position that is in conjugation with the image pick-up sensor 121 with respect to the half mirror 131. The pipe body 110 includes the branched pipe 140 for accommodating the light source 141 etc., and an internal space of the branched pipe 140 is communicated with an internal space of the pipe body 110. The light source 141 is, for example, an illuminating device configured of an LED panel in which high luminance LEDs are arranged two-dimensionally, and is driven by the circuit board 220. A portion of illumination light emitted from the light source 141 toward the half mirror 131 is reflected by the half mirror 131 to be guided to the tip end side of the pipe body 110 and is emitted from the objective lens 151 toward the object to be observed.

In this embodiment, the light source 141 also has a function as a detection light source that emits a detection light serving as a TOF (Time Of Flight) sensor in addition to a function as a light source of an illumination light that illuminates the object to be observed. Specifically, the light source 141 emits light that is obtained by superimposing, for example, the detection light which is an extremely short-pulsed light on a normal illumination light owing to the control performed by the control unit 200. Details of the TOF sensor are omitted since it is a well-known art. The detection light emitted from the light source 141 is reflected by the half mirror 131 in a manner similar to the manner in which the illumination light is reflected thereby and guided to the tip end side of the pipe body 110 and then projected toward the object to be observed from the objective lens 151. The detection light to be superimposed on the normal illumination light may not necessarily be a visible light as long as it is reflected by the half mirror 131 and projected on the object to be observed. For example, it may be a near-infrared light.

A half mirror 132 is disposed between the half mirror 131 and the light source 141 in a central vicinity of the branched pipe 140 in an oblique manner with respect to an optical axis of the branched pipe 140. The half mirror 132 is a spectroscopic optical device that reflects a portion of luminous flux reflected by the half mirror 131 to a light receiving sensor 122. Therefore, the detection light reflected by the object to be observed enters the internal space of the pipe body 110 again from the objective lens 151, is reflected by each of the half mirrors 131 and 132, and is condensed by a condensing lens 154 whereby it is made incident on the light receiving sensor 122. The detection light made incident on the light receiving sensor 122 is photoelectronically converted by the light receiving sensor 122 and is transmitted as a detection signal to the control unit 200 through the circuit board 220.

The control unit 200 is, for example, a CPU, and initiates image acquisition by the image pick-up sensor 121 or turns on the light source 141 by transmitting a control signal to the circuit board 220. Further, the control unit 200 executes autofocus at the time of image acquisition. Specifically, the illumination light is superimposed on the detection light with respect to the light source 141 and projected on the object to be observed. Then, the detection signal from the light receiving sensor 122 that has received the detection light reflected by the object to be observed is obtained. Time that has elapsed in the meantime, that is, the time taken from the emission of the detection light to the reception thereof is measured to calculate a distance to the object to be observed. In this case, the control unit 200 functions as a calculation unit that calculates a distance to the object to be observed.

Further, the control unit 200 calculates a moving position of the focus adjustment lens so that the object to be observed that is present at the calculated distance is brought into focus and controls the actuator so as to move the focus adjustment lens to the moving position. That is, the control unit 200 functions as a focus adjustment unit that adjusts the focus by moving the focus adjustment lens in the optical axis direction. The control unit 200 executes the autofocus as described above and makes the image pick-up sensor 121 perform the image pick-up operation. As described above, by performing autofocus adopting the TOF system, it is possible to realize swift autofocus that is less likely to involve wavering compared to autofocus adopting a contrast system (i.e., a hill-climbing AF system) that is restricted depending on the optical conditions. Further, an optical path of the detection light for both the projection and reception thereof is the same as the optical path of the observation image from the objective lens 151 to the image-forming lens 153, and thus the F value of the optical system can be made small. In other words, the diameter of the pipe body 110 can be made small.

The control unit 200 receives image data from the circuit board 220, converts the data into a display image signal, and displays it on the display unit 210. The display unit 210 is, for example, a display using an LCD panel, and displays the observation image of the object to be observed that is image-processed by the control unit 200. Note that in this embodiment, the rigid endoscope system 100 is described as including the control unit 200 and the display unit 210, however the rigid endoscope system may be configured so as to have the control unit 200 and the display unit connected as external devices as long as, for example, the circuit board 220 performs the functions of the calculation unit and the focus adjustment unit.

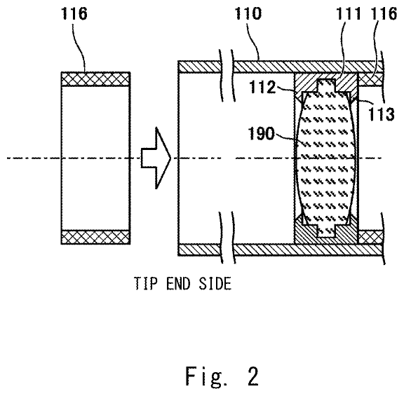

FIG. 2 is a diagram showing a state in which the optical system is accommodated inside the pipe body 110. Each of the lenses which is a single lens is supported by an annular support ring 111 that is loosely fitted to an inner wall of the pipe body 110. The support ring 111 is made of, for example, resin. The support ring 111 supports a lens 190 and has a thickness in the optical axis direction to an extent that does not involve an optical face tilt inside the pipe body 110.

In this embodiment, the illumination light that illuminates the object to be observed proceeds from the substrate edge side to the tip end side and the observation image is transmitted from the tip end side to the substrate side, and thus a stray light reduction process of reducing stray light is performed on both sides of the lens 190. Specifically, for example, a front-side aperture diaphragm 112 is disposed on the tip end side and a back-side aperture diaphragm 113 is disposed on the substrate edge side so as to surround an effective diameter of the lens 190. An edge cross section of each of the aperture diaphragms has a tapered part that opens in a direction receding from a surface of the lens 190. The front-side aperture diaphragm 112 and the back-side aperture diaphragm 113 may be formed integrally with the support ring 111 or may be formed as separate components and attached to the support ring 111.

An interval between the lenses is adjusted by interposing an adjustment ring 116 between the lenses. The adjustment ring 116 is an annular spacer that is loosely fitted to the inner wall of the pipe body 110. The adjustment ring 116 is made of, for example, resin.

The support ring 111 that supports the lens 190 and the adjustment ring 116 are inserted into the pipe body 110 in this order from the tip end side opening of the pipe body 110. The support ring 111 and the adjustment ring 116 that are adjacent to each other are mutually bonded, for example, by an adhesive. Through the assembly work described above, the lens configuration shown in FIG. 1 is realized. Note that the half mirrors 131 and 132, the image-forming lens 153, the image pick-up sensor 121, the light receiving sensor 122, the condensing lens 154, and the light source 141 etc. are assembled from the substrate edge side. Further, the support ring 111 may be structured so as to collectively support a plurality of lenses.

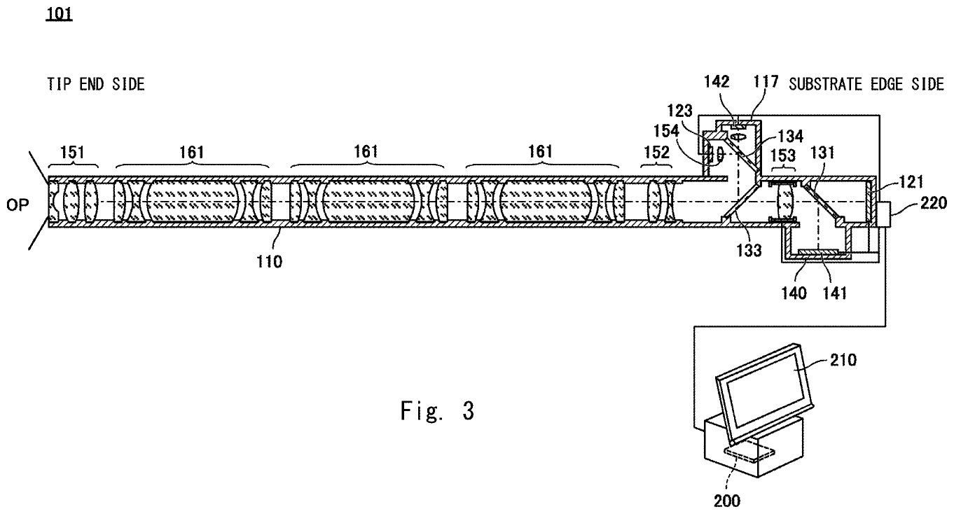

FIG. 3 is an overall schematic diagram of a rigid endoscope system 101 according to a second embodiment. Elements that are the same as those of the rigid endoscope system 100 according to the first embodiment are denoted by the same reference symbols as those mentioned in FIG. 1 and explanations thereof are omitted unless specifically referred to.

The rigid endoscope system 101 according to the second embodiment includes a floodlight LED 142 as a detection light source that is independent of the light source 141 of the illumination light. The floodlight LED 142 emits a detection light which is an infrared light owing to the control performed by the control unit 200. Accordingly, a light receiving sensor 123 has light receiving characteristics of an infrared region.

Specifically, a beam splitter 133 is disposed between the ocular lens 152 and the image-forming lens 153 in an oblique manner with respect to the optical axis OP. The beam splitter 133 is a spectroscopic optical device that allows a visible light to transmit therethrough and reflects an infrared light. A branch pipe 117 that is communicated with the pipe body 110 is disposed in the direction toward which the infrared light is reflected. The floodlight LED 142 and the light receiving sensor 123 are accommodated inside the branch pipe 117. A half mirror 134 is disposed between the beam splitter 133 and the floodlight LED 142 in a central vicinity of the branch pipe 117 in an oblique manner with respect to an optical axis of the branch pipe 117. The half mirror 134 is a spectroscopic optical device that reflects a portion of luminous flux reflected by the beam splitter 133 to the light receiving sensor 123.

The detection light emitted from the floodlight LED 142 is transmitted through the half mirror 134 and reflected by the beam splitter 133 to be guided to the tip end side and is projected toward the object to be observed from the objective lens 151. The detection light reflected by the object to be observed enters the internal space of the pipe body 110 again from the objective lens 151, is reflected by each of the beam splitter 133 and the half mirror 134, and is condensed by the condensing lens 154 thereby being made incident on the light receiving sensor 123. The detection light made incident on the light receiving sensor 123 is photoelectronically converted by the light receiving sensor 123 and is transmitted as a detection signal to the control unit 200 through the circuit board 220. The control unit 200 measures the time taken from the emission of the detection light by the floodlight LED 142 to the reception thereof by the light receiving sensor 123 to calculate a distance to the object to be observed. Further, the control unit 200 calculates a moving position of the focus adjustment lens so that the object to be observed that is present at the calculated distance is brought into focus and controls the actuator so as to move the focus adjustment lens to the moving position.

Even by the rigid endoscope system 101 configured as described above, it is possible to realize swift autofocus that is less likely to involve wavering than when the contrast system is adopted. Further, since the distance to the object to be observed is detected using the infrared light, it is possible to suppress the influence of the visible light on the picked-up image. Further, an optical path of the detection light for both the projection and reception thereof is the same as the optical path of the observation image from the objective lens 151 to the ocular lens 152, and thus the F value of the optical system can be made small. In other words, the diameter of the pipe body 110 can be made small. Note that the light source 141 of the illumination light is accommodated in the branched pipe 140 in the manner similar to the manner in which the rigid endoscope system 100 is accommodated, except that the half mirror 132, the condensing lens 154, and the light receiving sensor 122 are not disposed to the branched pipe 140.

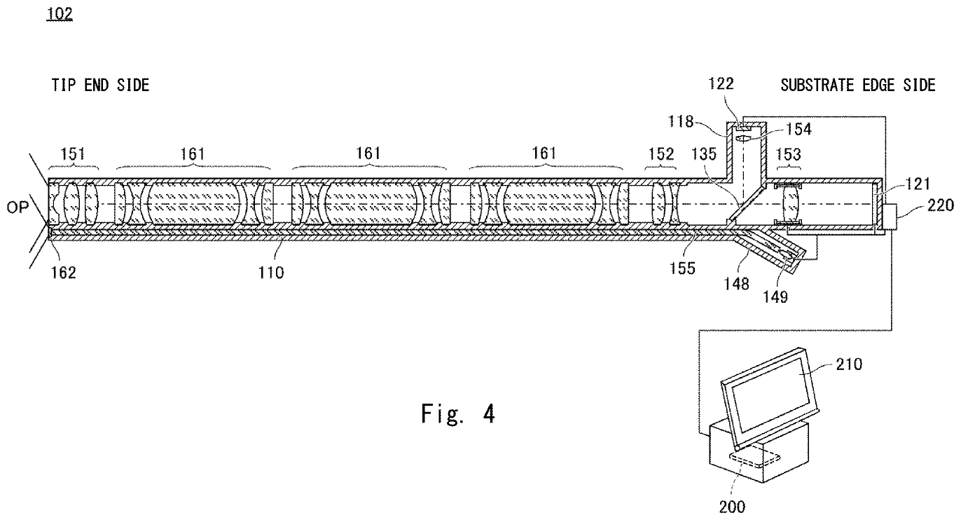

FIG. 4 is an overall schematic diagram of a rigid endoscope system 102 according to a third embodiment. Elements that are the same as those of the rigid endoscope system 100 according to the first embodiment and the rigid endoscope system 101 according to the second embodiment are denoted by the same reference symbols as those mentioned in FIGS. 1 and 3, and explanations thereof are omitted unless specifically referred to.

The rigid endoscope system 102 according to the third embodiment includes an optical fiber 155 as a light guide for the illumination light that illuminates the object to be observed. The optical fiber 155 is inserted into the internal space of the pipe body 110, and the illumination light that propagates within the optical fiber 155 is diffused from the tip end of the pipe body 110 and illuminates the object to be observed. Note that in the figure, one optical fiber 155 is shown, however, a plurality of optical fibers 155 may be disposed so as to surround a group of lenses within the internal space of the pipe body 110.

The substrate edge side of the of the optical fiber 155 is accommodated inside a branched pipe 148 and a projection light, which is emitted by a floodlight LED 149 that is also accommodated inside the branched pipe 148, is made incident from an edge surface thereof. The floodlight LED 142 also has a function as a detection light source that emits a detection light as the TOF sensor in addition to a function as a light source of an illumination light that illuminates the object to be observed similarly to the light source 141 of the rigid endoscope system 100. That is, the floodlight LED 149 emits light that is obtained by superimposing, for example, a detection light which is an extremely short-pulsed light on a normal illumination light owing to the control performed by the control unit 200. The detection light emitted from the floodlight LED 149 is guided by the optical fiber 155 along with the illumination light and projected toward the object to be observed from a diffusion lens 162.

A half mirror 135 is disposed between the ocular lens 152 and the image-forming lens 153 in an oblique manner with respect to the optical axis OP. The half mirror 135 is a spectroscopic optical device that transmits a portion of luminous flux through the ocular lens 152 side and reflects the rest of the luminous flux to a branch pipe 118 side. The branch pipe 118 is communicated with the pipe body 110 and accommodates therein the condensing lens 154 and the light receiving sensor 122.

The detection light reflected by the object to be observed enters the internal space of the pipe body 110 from the objective lens 151 and transmits through the group of lenses to be reflected by the half mirror 135. The detection light reflected by the half mirror 135 is condensed by the condensing lens 154 and made incident on the light receiving sensor 122. The detection light made incident on the light receiving sensor 122 is photoelectronically converted by the light receiving sensor 122 and is transmitted as a detection signal to the control unit 200 through the circuit board 220. The control unit 200 measures the time taken from the emission of the detection light by the floodlight LED 149 to the reception thereof by the light receiving sensor 122 to calculate a distance to the object to be observed. Further, the control unit 200 calculates a moving position of the focus adjustment lens so that the object to be observed that is present at the calculated distance is brought into focus and controls an actuator so as to move the focus adjustment lens to the moving position.

Even by the rigid endoscope system 102 configured as described above, it is possible to realize swift autofocus that is less likely to involve wavering than when the contrast system is adopted. Further, since the illumination light is propagated through the optical fiber 155, it is possible to suppress the influence of the stray light on the image compared to the case where the illumination light is passed through inside the group of lenses.

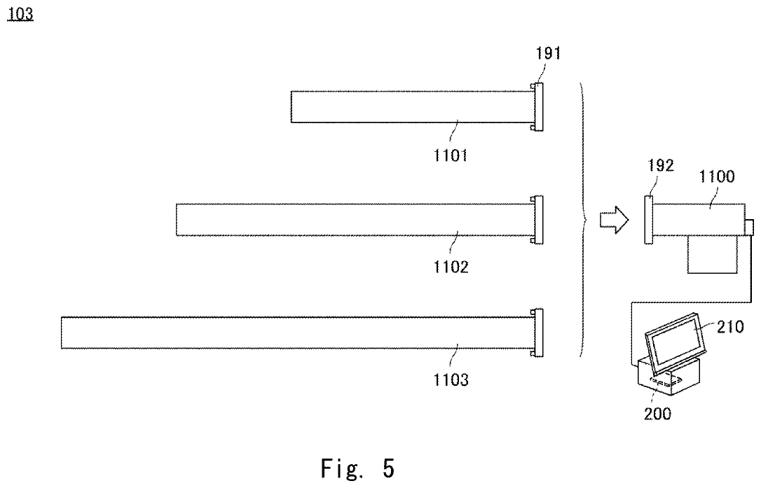

FIG. 5 is an overall schematic diagram of a rigid endoscope system 103 according to a fourth embodiment. In the rigid endoscope systems 100, 101, and 102 according to the first to third embodiments, the pipe body 110 that supports the objective lens 151 at the tip end side and supports the image pick-up sensor 121 at the substrate edge side has been integrally formed including the case where the branch pipe and the branched pipe are provided. In the rigid endoscope system 103, the pipe body 110 is configured such that a main pipe part, that supports the objective lens 151, the relay lens 161, and the ocular lens 152, and a base part, that accommodates the elements disposed on the substrate edge side of the ocular lens 152, are configured to be attachable/detachable.

Since the rigid endoscope system is used by inserting the tip end portion into a body of a test subject, it is preferable that the length of the main pipe part be selectable according to the depth of the insertion. Therefore, the rigid endoscope system 103 has the plurality of main pipe parts each having a different length from each other prepared in a replaceable manner as replacement pipe bodies. For example, as shown in the figure, a first main pipe 1101 that is relatively short, a second main pipe 1102 having a standard length, and a third main pipe 1103 that is relatively long are prepared. The main pipe part has a structure that is the same as that from the objective lens 151 to the ocular lens 152 of either one of the rigid endoscope systems 100, 101, or 102, according to the first to third embodiments and the length thereof is adjusted according to the number of pairs of the relay lenses 161 to be incorporated. That is, the first main pipe 1101, the second main pipe 1102, and the third main pipe 1103 have optical wavelengths different from each other.

The elements of the base part are accommodated in a pipe body base 1100. The base part has a structure that is the same as that on the substrate edge side of the ocular lens 152 of either one of the rigid endoscope systems 100, 101, or 102. Each of the first main pipe 1101, the second main pipe 1102, and the third main pipe 1103 includes a main pipe mount 191 at an end part on the substrate edge side. Further, the pipe body base 1100 includes a pedestal mount 192 at an end part on the tip end side. A user brings the rigid endoscope system 103 to a usable state by connecting the main pipe mount 191 of the selected main pipe part and the pedestal mount 192 of the base part in an opposing manner. As described above, since the user can select the main pipe part having the most suitable length according to the object to be observed, it is possible to enhance the usability and the diagnosis efficiency.

Further, even when either one of the rigid endoscope systems 100, 101, or 102 are employed for the rigid endoscope system 103, the system employed in detecting the distance to the object to be observed is the TOF system. Accordingly, the optical wavelength up to the object to be observed changes as the length of the main pipe part changes. Specifically, the optical wavelength is affected by the number of pairs of the relay lenses 161 when the detection light passes through the relay lens 161, is affected by the length of the optical fiber 155 when the detection light passes through the optical fiber 155, or is affected by the lens configurations of the lenses other than the relay lens when the lens configurations of the lenses other than the relay lens differ for each main pipe part. Therefore, the control unit 200 acquires information on which one of the main pipe parts has been mounted on the base part and calculates the distance to the object to be observed using a correction value corresponding to the main pipe part which has been mounted.

The control unit 200 acquires information on which one of the main pipe parts has been mounted on the base part by reading selected options that are input by the user through a touch panel superimposed on the display unit 210. Other various methods can be employed for the acquisition of this information. For example, it is possible to dispose a phase reading unit to each main pipe mount and have the pedestal mount 192 detect a phase of the phase reading unit so as to enable the control unit 200 to recognize which one of the main pipe parts has been mounted. Alternatively, by detecting a chart disposed at a known distance after the main pipe part has been mounted and performing calibration of the chart, it is possible to recognize which one of the main pipe parts has been mounted and further, a correction value thereof can be directly calculated.

The control unit 200 determines, after recognizing which one of the main pipe parts has been mounted, the correction value for calculating the distance by referring to a look-up table defining the selectable main pipe part and its correction value in matrix. By using this correction value, the control unit 200 calculates a precise distance to the object to be observed and moreover, realizes an autofocus of high precision.

The rigid endoscope systems according to the first to the fourth embodiments have been described above. In any one of the embodiments, a contrast AF utilizing sequential images output from the image pick-up sensor 121 may be used supplementary. For example, the rigid endoscope system may be configured so as to focus at an aimed focal point by moving the focus adjustment lens back and forth within a minute range after moving the focus adjustment lens in accordance with the calculated distance to the object to be observed. That is to say, the control unit 200 may be configured so as to correct the moving position of the focal adjustment lens to a position at which a contrast evaluation value of the image of the object to be observed becomes the highest. Further, in the fourth embodiment, the correction value defined in the look-up table may be corrected by performing the contrast AF for every predetermined period. Specifically, when it becomes necessary to move the focal adjustment lens by performing the contrast AF, the correction amount equivalent to the movement amount of the focal adjustment lens increases/decreases from the correction value in the look-up table. As described above, by utilizing the contrast AF, it is possible to cope with the deviation in the focus that occurs over time.

Note that if each of the rigid endoscope systems described above is configured such that the pipe body 110 can perform spectral dispersion at the rear part of the ocular lens 152, the user can directly observe the object to be observed by looking into the ocular lens 152. In this case, the configuration on the substrate edge side including the image pick-up sensor 121 may be made to be attachable/detachable to the pipe body 110 as a camera unit.

The first to fourth embodiments can be combined as desirable by one of ordinary skill in the art.

While the invention has been described in terms of several embodiments, those skilled in the art will recognize that the invention can be practiced with various modifications within the spirit and scope of the appended claims and the invention is not limited to the examples described above.

Further, the scope of the claims is not limited by the embodiments described above.

Furthermore, it is noted that, Applicant's intent is to encompass equivalents of all claim elements, even if amended later during prosecution.

What is claimed is:

1. A rigid endoscope system comprising:
a linear pipe body;
an objective lens provided at one end side of the pipe body;
an ocular lens provided at the other end side of the pipe body opposite to the one end side thereof;
an image pick-up sensor configured to output an image pick-up signal for generating image data;
a detection light source configured to emit a detection light to an object to be observed;
a light receiving sensor configured to receive the detection light that is reflected by the object to be observed and has passed through the inside of the pipe body;
a spectroscopic optical device configured to spectrally disperse a luminous flux emitted from the ocular lens in each of a direction toward the image pick-up sensor and a direction toward the light receiving sensor;
a calculation unit configured to calculate a distance to the object to be observed by measuring time taken from emission of the detection light from the detection light source to reception thereof by the light receiving sensor;
an image-forming optical system configured to form an image of a portion of the luminous flux emitted from the ocular lens on the image pick-up sensor; and
a focus adjustment unit configured to perform a focus adjustment by moving a focus adjustment lens included in the image-forming optical system in an optical axis direction based on the distance calculated by the calculation unit.

2. The rigid endoscope system according to claim 1, wherein an illumination light for illuminating the object to be observed is used as the detection light.

3. The rigid endoscope system according to claim 2, wherein the spectroscopic optical device is a half mirror.

4. The rigid endoscope system according to claim 1, further comprising an illumination light source configured to emit an illumination light for illuminating the object to be observed, wherein
the detection light is superimposed on the illumination light and projected on the object to be observed.

5. The rigid endoscope system according to claim 4, wherein
the detection light is an infrared light, and
the spectroscopic optical device is a beam splitter configured to spectrally disperse the luminous flux into a visible light and an infrared light.

6. The rigid endoscope system according to claim 1, wherein
at least a part of the pipe body is replaceable by one of a plurality of replacement pipe bodies having optical wavelengths different from each other; and
the calculation unit calculates the distance using a correction value corresponding to the replacement pipe body with which the part of the pipe body has been replaced.

7. The rigid endoscope system according to claim 1, wherein the focus adjustment unit corrects a moving position of the focus adjustment lens based on a contrast evaluation value of an image of the object to be observed calculated from the image pick-up signal.
